(19) United States
(12) Patent Application Publication
(10) Pub. No.: US 2025/0359834 A1
(43) Pub. Date: Nov. 27, 2025

(54) Smart Mammography Image Acquisition System and Method

(71) Applicant: GE Precision Healthcare LLC, Waukesha, WI (US)

(72) Inventors: Laurence Vancamberg, Poissy (FR); Razvan Iordache, Clamart (FR); Clément Jailin, Antony (FR); Ruben Sanchez de la Rosa, Antony (FR)

(21) Appl. No.: 18/672,351

(22) Filed: May 23, 2024

(51) Int. Cl. A61B 6/50; A61B 6/00; A61B 6/04; A61B 6/06; A61B 6/46

(52) U.S. Cl. CPC A61B 6/502; A61B 6/0414; A61B 6/06; A61B 6/4435; A61B 6/466; A61B 6/469; A61B 6/482; A61B 6/54

(57) ABSTRACT

According to an exemplary embodiment of the disclosure, an imaging system and method is provided for determining the need for generation of enhanced diagnostic images of a patient. The imaging system includes a radiation source operable at to emit radiation at multiple energy levels, a detector alignable with the radiation source, a controller operably connected to the radiation source and detector to generate image data in an imaging procedure, and a computer aided detection (CAD) system configured to analyze low energy (LE) images to locate regions of interest (ROI) and/or other triggering attributes, characteristics or findings within the object. Upon locating one or more triggering attributes, characteristics or findings within the LE images, the system and method can acquire one or more high energy (HE) images of the object, and can process the one or more LE images and the one or more HE images to form enhanced diagnostic images.

### FIELD OF THE DISCLOSURE

The present disclosure relates generally to medical imaging systems, including mammography systems and devices, and more specifically to system and methods for determining whether additional images of the patient for diagnostic purposes.

### BACKGROUND OF THE DISCLOSURE

Embodiments of the invention relate generally to X-ray medical imaging, and more particularly to devices, systems and methods employed to perform various imaging procedures, such as mammography imaging procedures including but not limited to spectral mammography (SM), such as 2D/3D dual-energy mammography exams, full-field digital mammography (FFDM) or digital breast tomosynthesis (DBT) mammography exams.

Spectral mammography (SM) is an X-ray imaging modality used to scan breasts for screening, diagnosis and/or interventional examinations that uses either multiple x-ray acquisition at different energy giving multiple images, or one x-ray acquisition with a energy discriminant detector that will be used to create multiple images. The effectiveness of spectral mammography is affected by numerous factors, one of which is the two-dimensional (2D) rendering of images obtained using SM.

Alternative systems to SM are also known for breast imaging. Some examples include full-field digital mammography (FFDM), which captures the image directly onto a flat-panel detector, computed radiography, which involves the use of a cassette that contains an imaging plate, or digital breast tomosynthesis (DBT). A digital breast tomosynthesis (DBT) or mammography-tomography (mammo-tomo) system is a dedicated mammography system that acquires several (e.g., tens of) angularly offset projection X-ray images and uses the resulting X-ray image data to reconstruct three-dimensional (3D) image datasets.

The 3D image datasets are used to form various volumetric representations of the imaged breast, including an entire 3D volume of the breast, and various 3D sections of the 3D volume, such as slices or slabs constituting specified thicknesses of the 3D volume oriented to provide the desired view of one or more regions of interest (ROI) detected within the 3D image dataset.

In addition, when the 3D image datasets of the breast have been produced, after being utilized in a suitable diagnosis procedure, they can be utilized to guide a biopsy device employed with the DBT system into the breast to obtain a biopsy of any one or more regions of interest (ROI) identified within the 3D image datasets. In DBT systems, the biopsy device is disposed directly on the DBT system in order to be able to perform the biopsy utilizing the 3D image dataset to guide the biopsy device to the ROI.

With regard to the use of mammography devices, the process of obtaining high quality mammographic images from breast tissue requires a technician to position the breast of a patient between one or more paddles that compress the breast in order to immobilize and flatten it during image acquisition. The compression force applied to a breast improves image quality by reducing the thickness of the breast while spreading the breast tissue over a larger area; this facilitates interpretation of obtained imagery since the amount of overlying tissue for structures within the imaged breast is minimized. Reduction of the breast thickness by compression is also important in managing patient radiation dosage. In general, the thicker the compressed breast, the more x-ray attenuation.

In some spectral mammography procedures, the images of the breast under compression on the imaging device are obtained using a low energy (LE) acquisition (tube voltage of the X-ray emitter of between about 20-40 kVp) and a high energy (HE) acquisition (tube voltage of the X-ray emitter of between about 45-80 kVp). The tissues of the breast have different attenuations of the LE and HE X-rays produced by the X-ray emitter, such that different tissues are represented more distinctly in the LE image data versus the HE image data, and vice versa. This is particularly true in the case of contrast enhanced (CE) imaging procedures, such as CE spectral mammography (CESM) and contrast-enhanced digital breast tomography (CE-DBT). If utilized in these procedures, the contrast agent, e.g., iodine, injected into the patient prior to the performance of the LE and HE imaging procedures can enhance the attenuation of numerous types of breast tissues constituting ROIs, including masses, calcifications, and cancerous tumors, among others, such that the ROIs are more clearly illustrated in the image data, and particularly within recombined images formed from the image data provided by the LE and HE acquisitions, whether contrast enhanced or not, in any of a number of known manners in order to produce high quality combined 2D and/or 3D images of the breast for diagnostic purposes.

With prior art mammography imaging devices and the procedures performed thereby, initially the breast is imaged to perform only LE acquisitions of the breast for producing LE images, such as in a FFDM or DBT imaging procedure. If the 2D or 3D LE images produced from the LE acquisitions are unable to provide sufficient information for making a confident diagnosis of the breast, the patient is rescheduled for a follow-up screening examination. In the follow-up examination the imaging procedure includes performing both LE and HE acquisitions of the breast in a SM mammography imaging procedure in order to provide the recombined image for diagnostic purposes.

While the selective performance of the follow-up imaging procedure with the LE and HE acquisitions performed together prevents the need for unnecessary HE acquisitions where the initial LE images alone are sufficient for diagnostic purposes, when required, the follow-up examination creates significant issues for the patient. More specifically, the follow-up examination is performed at a different date and time from the initial screening procedure, increasing the time for completion of the examination. Additionally, the follow-up examination requires an additional compression of and radiation dose delivered to the breast.

To avoid these issues concerning the follow-up examination, the LE and HE acquisitions of the breast can automatically be performed in the initial screening imaging procedure. This situation prevents the need for multiple compressions of the breast on the mammography imaging device, which can result in acquisitions being performed on the breast in different compressed positions on the mammography imaging device, and thus requiring more complex registration of the LE and HE image with one another in the formation of the combined 2D and/or 3D image. Further, each of the LE and HE acquisitions required for producing the recombined image for diagnostic purposes are performed in a single imaging procedure, greatly shortening the time required for completion of the diagnosis of the imaged breast.

However, as a result of performing the LE and HE acquisitions automatically in the initial screening mammography imaging procedure, the X-ray exposure dose of the breast is increased. Further, the breast compression time is also increased as a result of the HE acquisition being performed along with the LE acquistion. In situations where the diagnostic combined image shows no ROIs in the breast tissue, the HE acquisition does not provide significant enhancement to the diagnostic properties of the combined image, while increasing the X-ray exposure dose and time to the patient.

Therefore, it is desirable to develop a mammography imaging system and method that can selectively conduct HE acquisitions in situations where a suspicious ROI or other trigger has been identified in LE images but for which a HE acquisition is determined to be necessary to provide a diagnostic enhancement to the images produced by the mammography imaging system for clinical review.

### SUMMARY OF THE DISCLOSURE

According to an aspect of an exemplary embodiment of the present disclosure, a mammography system includes a radiation source operable at to emit radiation at multiple energy levels, a detector alignable with the radiation source, the detector having a surface on which an object to be imaged is adapted to be positioned, a controller operably connected to the radiation source and the detector to control the operation of the radiation source and detector to generate image data in an imaging procedure performed by the imaging system, the controller including a central processing unit and interconnected database for processing the image data from the detector to create images, a display operably connected to the controller for presenting information to a user, and a user interface operably connected to the controller to enable user input to the controller, wherein the controller is configured to acquire one or more low energy (LE) images of the object with the radiation source and the detector, to optionally acquire one or more additional images of the object after a determination of one or more triggering attributes, characteristics or findings in the object within the one or more LE images, the one or more additional images acquired with radiation having an energy level different than employed for acquiring the one or more LE images if one or more triggering attributes, characteristics or findings are located in the object, and to optionally process the one or more LE images and the one or more additional images to form one or more enhanced images.

According to still another aspect of an exemplary embodiment of the present disclosure, a method for determining the need for generation of enhanced diagnostic images of a patient includes the steps of providing an imaging system having a radiation source operable at to emit radiation at multiple energy levels, a detector alignable with the radiation source, the detector having a surface on which an object to be imaged is adapted to be positioned, a controller operably connected to the radiation source and the detector to control the operation of the radiation source and detector to generate image data in an imaging procedure performed by the imaging system, the controller including a central processing unit and interconnected database for processing the image data from the detector to create images, a display operably connected to the controller for presenting information to a user, and a user interface operably connected to the controller to enable user input to the controller, positioning the object on the surface between the radiation source and the detector, acquiring one or more low energy (LE) images of the object, analyzing the one or more LE images to locate one or more triggering attributes, characteristics or findings within the object, optionally acquiring one or more additional images of the object if one or more triggering attributes, characteristics or findings are located in the object, the one or more additional images acquired with radiation having an energy level different than employed for acquiring the one or more LE images, and optionally processing the one or more LE images and the one or more additional images to form one or more enhanced images.

These and other exemplary aspects, features and advantages of the invention will be made apparent from the following detailed description taken together with the drawing figures.

### BRIEF DESCRIPTION OF THE DRAWINGS

The drawings illustrate the best mode currently contemplated of practicing the present invention.

In the drawings:

FIG. 1 is an isometric view of an imaging system in the form of a mammography system for imaging the breast tissue of a patient, in accordance with an embodiment of the disclosure;

FIG. 2 is a diagram of the mammography device of FIG. 1, showing the radiation source of the mammography system in a scanning position, in accordance with an embodiment of the disclosure;

FIG. 3 is a flowchart illustrating the operation of the mammography system in accordance with a first embodiment of the disclosure.

FIG. 4 is a flowchart illustrating the operation of the mammography system in accordance with a second embodiment of the disclosure.

FIG. 5 is a flowchart illustrating the operation of the mammography system in accordance with a third embodiment of the disclosure.

FIG. 6 is a flowchart illustrating the operation of the mammography system in accordance with a fourth embodiment of the disclosure.

FIG. 7 is a flowchart illustrating the operation of the mammography system in accordance with a fifth embodiment of the disclosure.

FIG. 8 is a flowchart illustrating the operation of the mammography system in accordance with a sixth embodiment of the disclosure.

FIG. 9 is a flowchart illustrating the operation of the mammography system including a positioning assistance indicator in accordance with the disclosure.

FIG. 10 is a schematic view of the positioning assistance indicator of FIG. 9 on the mammography system in accordance with the disclosure.

### DETAILED DESCRIPTION OF THE DRAWINGS

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

As used herein, “electrically coupled”, “electrically connected”, and “electrical communication” mean that the referenced elements are directly or indirectly connected such that an electrical current may flow from one to the other. The connection may include a direct conductive connection, i.e., without an intervening capacitive, inductive or active element, an inductive connection, a capacitive connection, and/or any other suitable electrical connection. Intervening components may be present.

Further, while the embodiments disclosed herein are described with respect to a digital mammography apparatus for both 2-dimensional (2D) and 3-dimensional (3D) imaging of breast tissue including spectral mammography (single or multi-energy), it is to be understood that embodiments of the invention may be applicable to other types of imaging devices for either or both 2D and 3D imaging including, for example, fluoroscopy, full-field digital mammography (FFDM), and digital breast tomosynthesis (DBT), as well as for imaging procedures for tissues other than breast tissue. Further still, embodiments of the invention may be used to analyze tissue, generally, and are not limited to analyzing human tissue.

During imaging procedures using a digital mammography system, the breast of a patient is compressed and an x-ray source may be rotated around the breast within a range of angles in positive and negative directions from a medial position. Certain imaging procedures, including spectral mammography (SM) performed with a digital mammography system produce a dual energy image of the breast. A dual energy image may be generated from two images, where the two images include a first image acquired with low radiation energy (termed a low energy image, or LE) and a second image acquired with high radiation energy (termed a high energy image, or HE). A digital recombination process may be used to generate one or more dual energy images (DE). In certain dual energy imaging procedures, including contrast-enhanced spectral mammography (CESM), the visualization of one or more regions of interest (ROIs) in the dual energy image can be enhanced by the administration of a contrast agent, such as iodine, to an imaging subject (e.g., patient). For CESM the recombination process could be a digital subtraction such that background features are removed from the DE image and the one or more regions of interest (ROIs) (e.g., the lesion) are more clearly visualized. For breast compositional imaging the output of the recombination process is three DE images representing water lipid protein content of the breast, as describe in Laidevant A. D. et al., “Compositional breast imaging using a dual-energy mammography protocol”, Medical Physics, January 2010, which is expressly incorporated herein by reference I tis entirety for all purposes.

In certain dual energy imaging procedures, including contrast-enhanced spectral mammography (CESM), the visualization of one or more ROIs in the dual energy image can be enhanced by the administration of a contrast agent, such as iodine, to an imaging subject (e.g., patient).

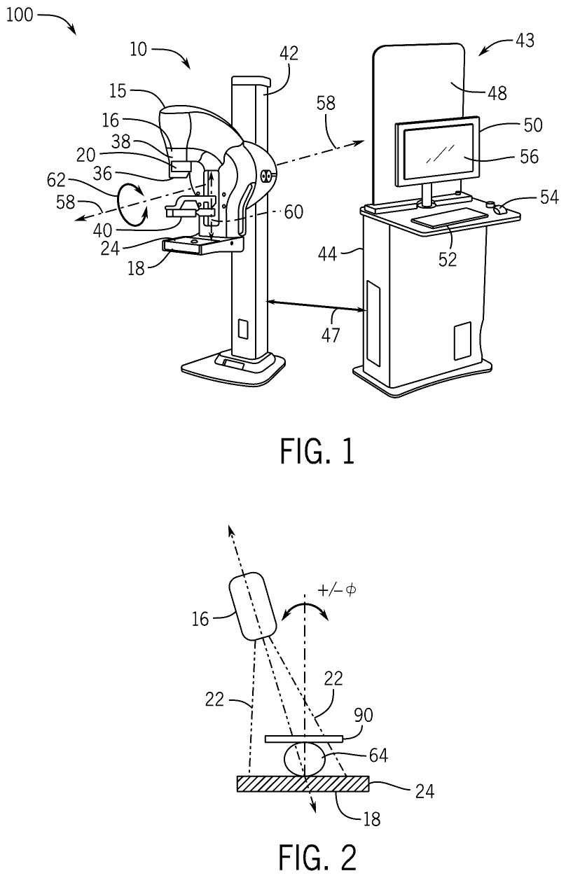

Referring to FIG. 1, a digital mammography system 100, such as that disclosed in US Patent Application Publication No. US2024/0074718, entitled Methods and Systems For Digital Mammography Imaging, the entirety of which is expressly incorporated by reference herein for all purposes, is shown including an x-ray system 10 for performing a mammography procedure, according to an embodiment of the disclosure.

The x-ray system 10 includes a support structure 42, to which a radiation source 16, a radiation detector 18, and a collimator 20 are attached. The radiation source 16 is housed within a gantry 15 that is movably coupled to the support structure 42. In particular, the gantry 15 may be mounted to the support structure 42 such that the gantry 15 including the radiation source 16 can rotate around an axis 58 in relation to the radiation detector 18. An angular range of rotation of the gantry 15 housing the radiation source 16 indicates a rotation up to a desired degree in either direction about the axis 58, as indicated by arrow 62. For example, the angular range of rotation of the radiation source 16 may be −θ to +θ, where θ may be such that the angular range is a limited angle range, less than 360 degrees. An exemplary x-ray system may have an angular range of ±11 degrees, which may allow rotation of the gantry (that is rotation of the radiation source) from −11 degrees to +11 degrees about an axis of rotation of the gantry. The angular range may vary depending on the manufacturing specifications. The angular range for digital mammography systems may be approximately +11 degrees to +60 degrees, depending on the manufacturing specifications.

The radiation source 16 is directed toward a volume or object to be imaged and is configured to emit radiation rays at desired times to acquire one or more images. The radiation detector 18 is configured to receive the radiation rays via a surface 24. The detector 18 may be any one of a variety of different detectors, such as an x-ray detector, digital radiography detector, or flat panel detector. The collimator 20 is disposed adjacent to the radiation source 16 and is configured to adjust an irradiated zone of a subject.

In some embodiments, the system 10 may further include a patient shield 36 mounted to the radiation source 16 via face shield rails 38 such that a patient's body part (e.g., head) is not directly under the radiation. The system 10 may further include a compression paddle 40, which may be movable upward and downward in relation to the support structure along a vertical axis 60. Thus, the compression paddle 40 may be adjusted to be positioned closer to the radiation detector 18 by moving the compression paddle 40 downward toward the detector 18, and a distance between the detector 18 and the compression paddle 40 may be increased by moving the compression paddle upward along the vertical axis 60 away from the detector. The movement of the compression paddle 40 may be adjusted by a user via compression paddle actuator (not shown) included in the x-ray system 10. The compression paddle 40 may hold a body part, such as a breast, in place against the surface 24 of the radiation detector 18. The compression paddle 40 may compress the body part and hold the body part still in place while optionally providing apertures to allow for insertion of a biopsy needle, such as a core needle or a vacuum assisted core needle. In this way, compression paddle 40 may be utilized to compress the body part to minimize the thickness traversed by the x-rays and to help reduce movement of the body part due to the patient moving. The x-ray system 10 may also include an object support (not shown) on which the body part may be positioned.

The digital mammography system 100 may further include a workstation 43 comprising a controller 44 including at least one processor and a memory. The controller 44 may be communicatively coupled to one or more components of the x-ray system 10 including one or more of the radiation source 16, radiation detector 18, the compression paddle 40, and a biopsy device. In an embodiment, the communication between the controller and the x-ray system 10 may be via a wireless communication system. In other embodiments, the controller 44 may be in electrical communication with the one or more components of the x-ray system via a cable 47. Further, in an exemplary embodiment, as shown in FIG. 1, the controller 44 is integrated into the workstation 43. In other embodiments, the controller 44 may be integrated into one or more of the various components of the system 10 disclosed above. Further, the controller 44 may include processing circuitry and associated electronic memory devices that execute stored program logic and may be any one of different computers, processors, controllers, or combination thereof that are available for and compatible with the various types of equipment and devices used in the x-ray system 10.

The workstation 43 may include a radiation shield 48 that protects an operator of the system 10 from the radiation rays emitted by the radiation source 16. The workstation 43 may further include a user interface 50, formed of one or more of a keyboard 52, mouse 54, and/or other appropriate user input devices that facilitate control of the system 10 via a user interface 50, as well as an interconnected display 56, which can also function as the user interface 50.

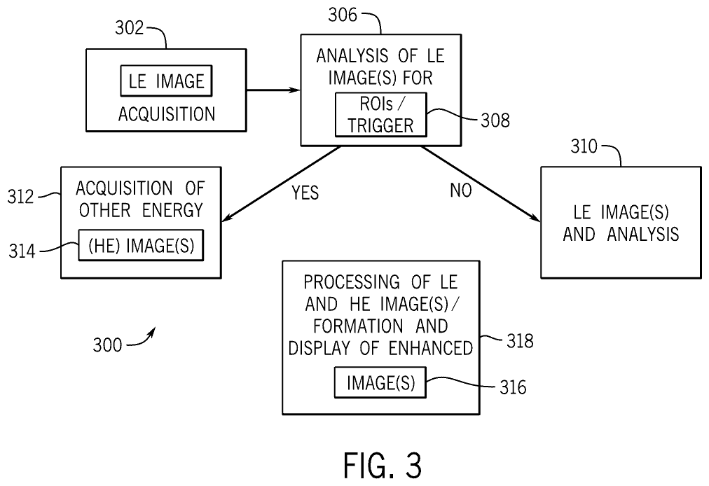

The controller 44 may adjust the operation and function of the x-ray system 10. As an example, the controller 44 may provide timing control, as to when the x-ray source 16 emits x-rays, and may further adjust how the detector 18 reads and conveys information or signals after the x-rays hit the detector 18, and how the x-ray source 16 and the detector 18 move relative to one another and relative to the body part being imaged. The controller 44 may also control how information, including images 45 presented on display 50 and data acquired during the operation, is processed, displayed, stored, and manipulated. Various steps of the method of operation of the x-ray system 10 and processing of the image data obtained thereby as described herein with respect to FIGS. 3-8 performed by the controller 44, may be provided by a set of instructions stored in non-transitory memory of the controller 44.

Further, as stated above, the radiation detector 18 receives the radiation rays 22 emitted by the radiation source 16. In particular, during imaging with the x-ray system, a projection image of the imaging body part may be obtained at the detector 18. In some embodiments, data, such as projection image data, received by the radiation detector 18 may be electrically and/or wirelessly communicated to the controller 44 from the radiation detector 18. The controller 44 may then reconstruct one or more scan images based on the projection image data, by implementing a reconstruction algorithm, for example. The reconstructed image may be displayed to the user on the user interface 50 via a display screen 56.

The radiation source 16, along with the radiation detector 18, forms part of the x-ray system 10 which provides x-ray imagery for the purpose of one or more of screening for abnormalities, diagnosis, dynamic imaging, and image-guided biopsy. For example, the x-ray system 10 may be operated in a mammography mode for screening for abnormalities. During mammography, a patient's breast is positioned and compressed between the detector 18 and the compression paddle 40. Thus, a volume of the x-ray system 10 between the compression paddle 40 and the detector 18 is an imaging volume. The radiation source 16 then emits radiation rays on to the compressed breast, and a projection image of the breast is formed on the detector 18. The projection image may then be reconstructed by the controller 44, and displayed on the interface 50. During mammography, the gantry 15 may be adjusted at different angles to obtain images at different orientations, such as a cranio-caudal (CC) image and a medio-lateral oblique (MLO) image. In one example, the gantry 15 may be rotated about the axis 58 while the compression paddle 40 and the detector 18 remain stationary. In other examples, the gantry 15, the compression paddle 40, and the detector 18 may be rotated as a single unit about the axis 58.

Further, the x-ray system 10 may be operated in a tomosynthesis mode for performing digital breast tomosynthesis (DBT). During tomosynthesis, the x-ray system 10 may be operated to direct low-dose radiation towards the imaging volume (between the compression paddle 40 and the detector 18) at various angles over the angular range of the x-ray system 10. Specifically, during tomosynthesis, similar to mammography, the breast is compressed between the compression paddle 40 and the detector 18. The radiation source 16 is then rotated from −θ to +θ, and a plurality of projection images of the compressed breast is obtained at regular angular intervals over the angular range. For example, if the angular range of the x-ray system is ±11 degrees, 22 projection images may be captured by the detector during an angular sweep of the gantry at approximately one every one degree, generating a set of angulated x-ray images. The plurality of projection images are then processed by the controller 44 to generate a plurality of DBT image slices. The processing may include applying one or more reconstruction algorithms to reconstruct three dimensional image of the breast. Furthermore, the x-ray system may be configured to perform a DBT-guided biopsy procedure. Accordingly, in some exemplary embodiments, the system 10 may further include a biopsy device comprising a biopsy needle for extracting a tissue sample for further analysis.

In some examples, including dual-energy 3D or stereotactic procedures, such as spectral mammography (SM), low-energy (LE) and high-energy (HE) image acquisitions are performed of the breast or other tissue with at least two different positions of the x-ray source with respect to the detector. The images are then recombined to display material-specific information with regard to the internal structure of the tissue being imaged.

In other embodiments, contrast agents can be optionally coupled with images taken using dual-energy imaging processes and technology. The contrast agents are taken up in the blood vessels surrounding a cancerous lesion in the breast and/or ROI, thereby providing a contrasting image for a period of time with respect to the surrounding tissue, enhancing the ability to locate the lesion.

In particular, contrast enhanced spectral mammography (CESM) (2D) and contrast enhanced digital breast tomosynthesis (CE-DBT) (3D) imaging modalities are performed with dual-energy technology. For each view (single view in CESM, multiple views for CE-DBT), a pair of images is acquired: a low-energy (LE) image and a high-energy (HE) image. After the injection of contrast medium, dual-energy images are acquired at each of two or more positions of the x-ray tube with respect to the detector. For each of these tube angulations, the low and high-energy images are recombined to produce an image of the contrast medium surface concentration at each pixel to provide an iodine-equivalent or dual-energy (DE) image(s) (for a single view in CESM, and for multiple views for CE-DBT), which in CE-DBT, are used to reconstruct a 3D volume. Image recombination may be performed based on simulations of the x-ray image chain, via calibrations on a reference phantom, or any other suitable 3D-reconstruction process. Additionally, in the continuous mode of acquisition where the x-ray tube moves continuously with interleaved HE and LE images being taken, the LE images are used to reconstruct a LE 3D volume, and the HE images are used to reconstruct a HE 3D volume, with both volumes being recombined in a suitable manner to provide an iodine 3D volume. In some examples, 3D-reconstruction and HE/LE recombination may be performed in a single step.

In CE-DBT, non-paired HE and LE images may be acquired for each view and an HE volume, LE volume, and recombined CE volumes may be reconstructed for the ROI. For example, the HE and LE views may be interleaved during the CE-DBT scan (alternatively HE, LE, HE, LE, HE, LE, etc.) with a switch from HE to LE then to HE again etc., for each angulated position of the x-ray tube. The LE and HE images are usually obtained at mean energies above and below the k-edge of the contrast agent. At x-ray energies just above the k-edge of the contrast agent, the absorption of x-rays is increased resulting in an increase of contrast from the iodine contrast agent in the HE image.

Referring now to FIG. 2, in operation of the x-ray system 10 in accordance with an embodiment, the breast 64 of the patient may be placed onto the surface 24 of the radiation detector 18. The compression paddle 40, under control of the controller 44, moves towards the detector 18 to compress the breast 64 against the surface 24 of the detector 18 such that the breast 64 is immobilized. Movement of the compression paddle 40 towards the detector 18 to compress the breast 64 against the support plate/detector 18 defines a compression phase of the x-ray system 10. Once a target compression is achieved, movement of the compression paddle 40 is halted and the compression paddle 40 and the detector 18 are held in fixed position to clamp the breast 64 therebetween (referred to herein as the clamping phase) so that the imaging or other procedures, e.g., a biopsy, may be commenced. During an imaging procedure, the radiation source 16 is selectively adjusted such that it is moved/rotated to a first scanning position and scans the breast 64. The radiation detector 18 receives the radiation rays 22 passing through the breast 64 and sends data to the controller 44 which then generates one or more x-ray images of the breast 64.

Looking now at FIG. 3, a first exemplary embodiment of a method 300 of operation of the mammography system 100 in accordance with the present disclosure is illustrated. The method 300 is employed on the mammography system 100 to selectively determine from one or more LE images acquired by the x-ray system 10 whether the acquisition of one or more additional images using radiation of a different energy than that employed for the LE images is required to provide enhanced images processed from the LE images and the other energy or additional images with clinical diagnostic information for review. In various embodiments, the other energy or additional images are acquired with radiation having a higher energy than that employed for the acquisition of the LE images.

In the method 300, after compression of the breast on the x-ray system 10 between the detector 18 and the compression paddle 40, in step 302 one or more LE images 304 are obtained of the breast 64, optionally in conjunction with the administration of a contrast agent. In an exemplary embodiment, the LE images 304 can be 2D or 3D images formed from image data obtained as a part of a mammography/FFDM (2D) or DBT (3D) screening imaging procedure. In step 306, the LE images 304 are analyzed by a user of the x-ray system 10, such as when presented to the user on the display 56, or by computer aided diagnosis (CAD) program or system contained within and/or as part of the controller 44 to determine the presence of any triggering information/triggers regarding the breast 64 within any of the LE images 304. The CAD program can be any suitable form of CAD analysis program, such as an artificial intelligence/deep learning program designed for detection and/or classification of one or more various attributes, characteristics or findings for the breast 64 within the LE images, and can provide information from the LE images 304 regarding any attributes, characteristics and/or findings that will benefit from/be enhanced by images obtained with HE acquisitions. Any one or more of these attributes, characteristics or findings will constitute a trigger for the subsequent acquisition of HE images of the breast 64 in order to provide further information on the breast 64 in relation to the attributes, characteristics or findings causing the HE image acquisition(s). In certain exemplary embodiments, the triggering attributes, characteristics or findings that constitute the trigger for the subsequent other energy/HE image acquisition(s) can include one or more ROI(s) 308, e.g., cysts, solid masses, etc. In other exemplary embodiments the triggering attributes, characteristics or findings that constitute the trigger for the subsequent HE image acquisition(s) can include, separately from or in addition to an ROI 308, other suspicious or non-suspicious attributes, characteristics or findings of specific portions of or the entirety of the breast 64. These other suspicious or non-suspicious attributes, characteristics or findings can include one or more of a tissue density classification or score of the entire breast 64 or portions thereof, or a risk factor score determined by the CAD program. The CAD program can additionally provide as outputs the determined attributes, characteristics or findings, such as a BI-RADS score for the entire breast and/or the one or more ROIs 308 identified in the LE images 304, that are employed as the trigger for manually and/or automatically performing subsequent HE acquisitions. The analysis provided by the CAD program/controller 44 can be performed in real time to provide the results of the analysis to the user as feedback during the screening imaging procedure. If no ROIs 308 or other triggers are determined to be present in the LE images 304, the method 300 proceeds to step 310 to present the finding of no ROIs 308 or other triggers within the LE images 304 and terminates the screening imaging procedure.

Alternatively, should the user or controller 44 locate a ROI 308 or other trigger(s) in the LE image(s) 304, in step 312 the x-ray system 10 is operated to perform a subsequent imaging procedure or acquisition at another energy level, e.g., an energy level higher than that used for acquisition of the LE mages 304. In one exemplary embodiment of the present disclosure, in step 312 the image acquisition produces HE images 314. The HE images 314 are obtained in step 312 with the breast 64 under the same compression as employed for the acquisition of the LE images 304 in step 302, such that the LE images 304 and HE images 314 are acquired with the breast 64 in the same position. After acquisition of the HE images 314 or other modality images, in step 318 the controller 44 processes the LE images 304 and the HE images 314 to form enhanced images in the form of a recombined 2D image and/or reconstructed 3D image 316 clearly presenting the location and other characteristics of the ROI(s) 308 for diagnostic review by the user and/or reviewing physician.

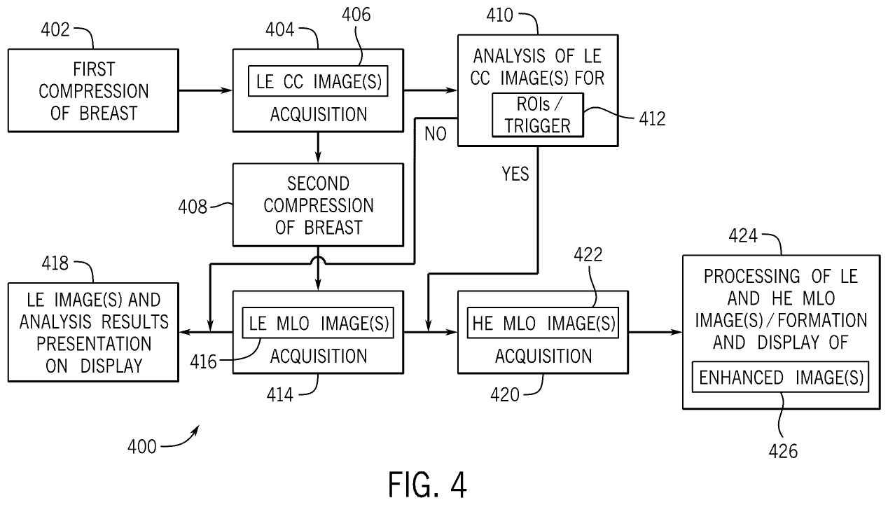

Looking now at FIG. 4, in a second exemplary embodiment of the disclosure, the method 400 involves the positioning of the breast 64 in step 402 between the compression paddle 40 and the detector 18 in position to acquire a cranial-caudal (CC) image of the breast 64. Once compressed, in step 404, the x-ray system 10 is operated to obtain a LE CC image 406 of the breast 64. Subsequently, in step 408, the breast 64 is released, repositioned and recompressed to obtain a mediolateral oblique (MLO) image of the breast 64. Alternatively, in other embodiments the LE MLO image 416 can be acquired prior to the LE CC image 406, such that the analysis by the CAD/controller 44 can be performed on the LE MLO image 416, and/or on the later acquired LE CC image 406. Concurrently with the decompression and repositioning of the breast 64 on the x-ray system 10 for the MLO view acquisition, in step 410 the LE CC image 406 is analyzed by the user and/or CAD/controller 44 to determine the presence of any ROIs 412 or other triggers within the LE CC image 406. Further, during and/or subsequent to the analysis of the LE CC image 406, in step 414, the x-ray system 10 is operated to acquire an LE MLO image 416 of the breast 64.

If no ROIs 412 or other triggers are located in the LE CC image 406, the method 400 proceeds to step 418 to present the finding of no ROIs 412 or other triggers within the LE CC image 406 along with the individual or combined LE CC image 406 and LE MLO image 416 and terminates the screening imaging procedure. Alternatively, if an ROIs 412 and/or other trigger is found in the LE CC image 406, in step 420 the x-ray system 10 is operated to obtain a HE MLO image 422 of the breast 64 under the same compression as for the LE MLO image 416. Subsequently, in step 424 the HE MLO image 422 is recombined with the LE MLO image 416, and optionally the LE CC image 406, to form a recombined or reconstructed 2D or 3D image 426 clearly presenting the location and other characteristics of the ROI(s) 412 and/or triggering attributes, characteristics or findings for diagnostic review by the user and/or reviewing physician, optionally with the LE CC image 406, the LE MLO image 416 an/or the HE MLO image 422.

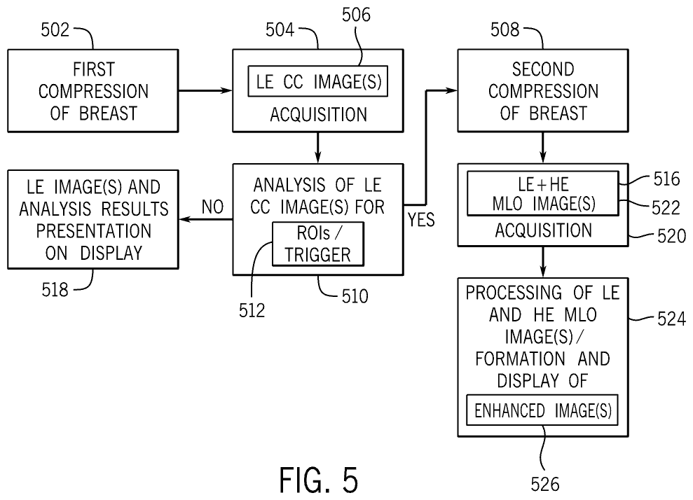

Referring now to FIG. 5, in a third exemplary embodiment of the disclosure, the method 500 involves the positioning of the breast 64 in step 502 between the compression paddle 40 and the detector 18 in position to acquire a cranial-caudal (CC) image of the breast 64. Once compressed, in step 504, the x-ray system 10 is operated to obtain a LE CC image 506 of the breast 64. Subsequently, in step 510 the LE CC image 506 is analyzed by the user and/or CAD/controller 44 to determine the presence of any ROIs 512 or other triggers within the LE CC image 506.

If no ROIs 512 or other triggers are located in the LE CC image 506, the method 500 proceeds to step 518 to present the finding of no ROIs 512 or other triggers within the LE CC image 506 and to operate the x-ray system 10 obtain LE MLO images of the breast 64 and/or terminate the screening imaging procedure. Alternatively, if an ROI 512 and/or triggering attribute, characteristic or finding is found in the LE CC image 506, in step 508 the breast 64 is released, repositioned and recompressed to obtain a mediolateral oblique (MLO) image of the breast 64. Afterwards, in step 520 the x-ray system 10 is operated to obtain both an optional LE MLO image 516 and a HE MLO image 522 of the breast 64 under the same compression.

Subsequently, in step 524 the HE MLO image 522 is recombined with the LE MLO image 516, and optionally the LE CC image 506, to form a combined or reconstructed 2D or 3D image 526 clearly presenting the location and other characteristics of the ROI(s) 512 and/or triggering attributes, characteristics or findings for diagnostic review by the user and/or reviewing physician, optionally along with the LE CC image 506, the LE MLO image 516 and/or the HE MLO image 522.

In any of the prior or subsequently described embodiments of the present disclosure, the type of imaging process or acquisition initially performed on the breast 64 to obtain the LE image(s) can be the same as the process subsequently performed on the breast 64 upon determination of one or more ROIs within the LE image(s) to acquire the HE image(s). For example, an initial LE 2D image acquisition can be followed by a HE 2D image acquisition. Further, an initial LE 3D or DBT image acquisition can be followed by a HE 3D or DBT image acquisition.

However, in other exemplary embodiments the imaging process utilized in the LE imaging acquisition and the HE imaging acquisition can be different from one another. For example, an initial LE 3D or DBT image acquisition can be followed by a HE 2D image acquisition. Additionally, an initial LE 2D image acquisition can be followed by a HE 3D or DBT image acquisition.

Further, in any of the prior or subsequently described embodiments of the present disclosure, in the step of the method 300,400,500 where the HE image is obtained, the x-ray system 10, can be operated to adjust the collimator 20, or other applicable radiation focusing mechanism or device, to focus the HE rays 22 onto an area including the ROI 306,412,512. In this manner, the image data utilized to form the HE image 314,422,522 provides enhanced resolution on the ROI(s) 306,412,512 and/or other triggering attributes, characteristics or findings in the breast 64 represented within the HE image 314,422,522 to provide the reconstructed 2D or 3D image 316,426,526 with highly detailed information regarding the location and other characteristics of the ROI(s) 306,412,512 and/or other triggering attributes, characteristics or findings within the breast 64 for diagnostic review by the user and/or reviewing physician.

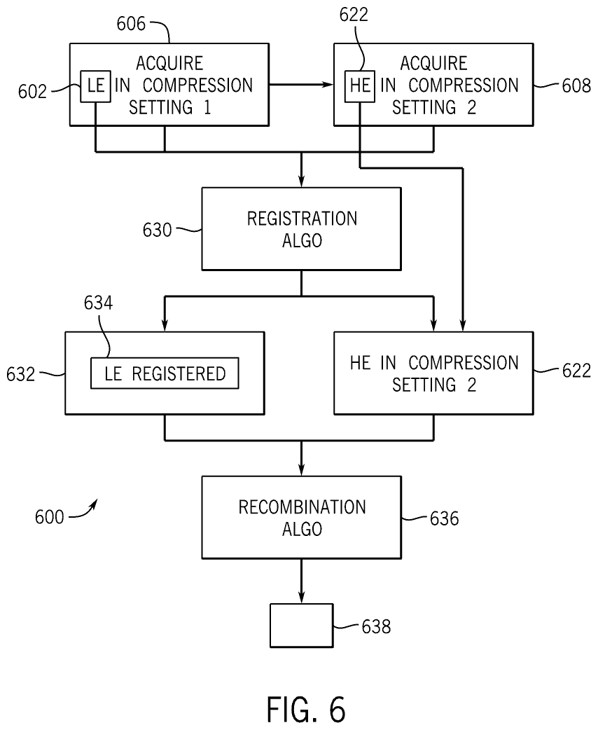

Looking now at FIG. 6, in a fourth exemplary embodiment of the disclosure, in certain embodiments the LE image in which the ROI and/or other triggering attributes, characteristics or findings in the breast 64 is detected and the HE image for the same view are obtained at different compressions of the breast 64, i.e., the breast 64 is decompressed, repositioned and recompressed between the LE image acquisition that is analyzed and the HE image acquisition for the same view. In these situations, prior to the recombination of the LE image with the HE image to provide the recombined or reconstructed image for diagnostic review of ROIs present in the breast and/or other triggering attributes, characteristics or findings in the breast 64, the LE image and HE image must be registered to one another.

In one example of this, a method 600 shown in FIG. 6 includes an initial step 602 where the breast 64 is positioned between the compression paddle 40 and the detector 18 in a first compression/position to acquire a LE image 606 of the breast 64. The position of the breast 64 for the LE image 606 can be for one of a CC view or an MLO view. Once compressed, in step 602, the x-ray system 10 is operated to obtain the LE image 606 of the breast 64 that is analyzed to determine the presence of any ROIs and/or other triggering attributes, characteristics or findings in the breast 64 within the LE image 606. Subsequently, in the case where one or more ROIs and/or other triggering attributes, characteristics or findings in the breast 64 are located within the LE image 606, in step 608, after the breast 64 has been released or decompressed, such as to acquire other images of different views of the breast 64 or after termination of an earlier imaging procedure, the breast 64 is repositioned and recompressed in the x-ray device 10 in a second compression/position to obtain the other of the HE image 622 of the same CC or MLO view of the breast 64 for the LE image 606. The second compression is any position and compression of the breast 64 used to obtain a HE image 622 of the breast 64 after releasing the breast 64 from the position and compression used to acquire the LE image 606, as the position and compression for the HE image 622 will not be identical to that used for the LE image 606, i.e., the first compression. After acquiring the HE image 622 at the second compression, in step 630 the LE image 606 obtained at the first compression and the HE image 622 obtained at the second compression are registered to form a registered image 632, which in the illustrated embodiment is a registered LE image 634. The algorithm utilized for the registration of the LE image 606 to the HE image 622 is any suitable registration algorithm, such as that disclosed in Clément Jailin et al 2023 Biomed. Phys. Eng. Express 9 035003, the entirety of which is expressly incorporated by reference herein for all purposes, and can be included as a part of the controller 44 or as a set of executable instructions stored on the workstation and accessible by the controller 44 in order to perform the registration step 630. The registered LE image 634 and the HE image 622 can subsequently be recombined or reconstructed in step 636 into a recombined 2D or 3D image 638 including information regarding the location and other characteristics of the ROI(s) therein and/or other triggering attributes, characteristics or findings in the breast 64 for diagnostic review by the user and/or reviewing physician.

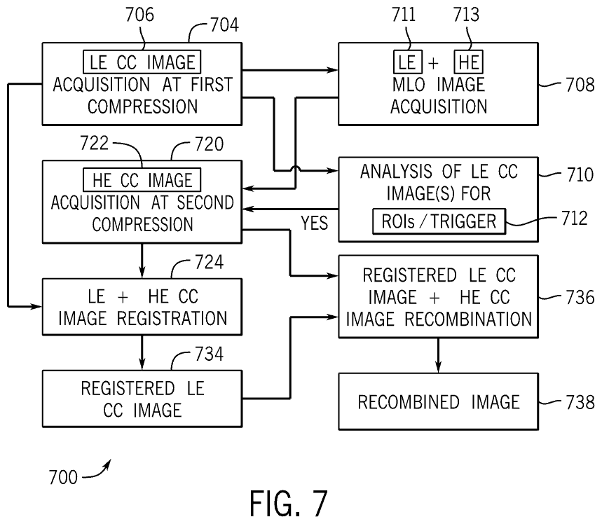

In one particular exemplary embodiment of the method of FIG. 6, as shown in the fifth exemplary embodiment in FIG. 7 the method 700 involves the positioning of the breast 64 between the compression paddle 40 and the detector 18 at a first compression in a position to acquire a cranial-caudal (CC) image 706 of the breast 64 in step 704 with the x-ray system 10. Subsequently, in step 708, the breast 64 is released, repositioned and recompressed to obtain an MLO image, e.g., a LE MLO image 711 and/or a HE MLO image 713, of the breast 64. Concurrently with the release, repositioning and recompression of the breast 64 on the x-ray system 10 for the MLO view(s) acquisition, in step 710 the LE CC image 706 is analyzed by the user and/or CAD/controller 44 to determine the presence of any ROIs 712 and/or other triggering attributes, characteristics or findings in the breast 64 within the LE CC image 706.

If an ROI 712 and/or other triggering attributes, characteristics or findings in the breast 64 is found in the LE CC image 706, in step 720 the breast 64 is released, repositioned and recompressed in a second compression to obtain an HE CC image 722 of the breast 64. Subsequently, in step 724 the LE CC image 706 and HE CC image 722 are registered to one another to form a registered LE image 734. In step 736 the registered LE CC image 734 is recombined with the HE CC image 722 to form a recombined or reconstructed 2D or 3D image 738 clearly presenting the location and other characteristics of the ROI(s) 712 and/or other triggering attributes, characteristics or findings in the breast 64 for diagnostic review by the user and/or reviewing physician.

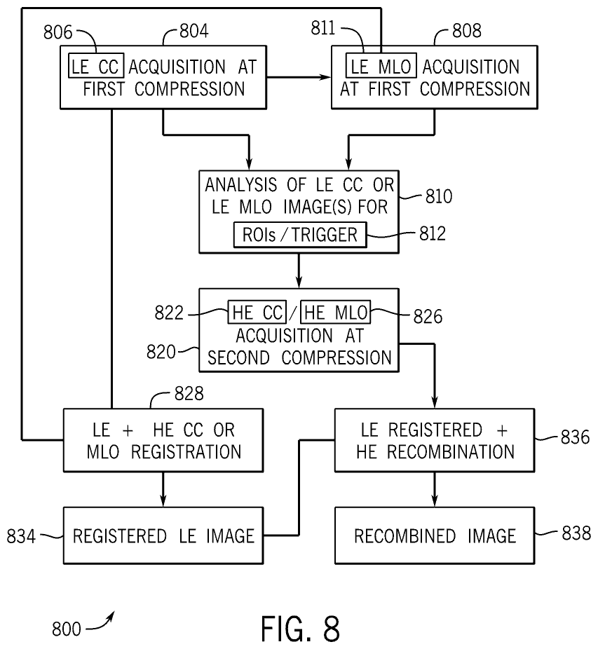

In another particular exemplary embodiment of the method of FIG. 6, as shown in the sixth exemplary embodiment in FIG. 8 the method 800 involves the positioning of the breast 64 between the compression paddle 40 and the detector 18 at a first compression in a position to acquire a cranial-caudal (CC) image 806 of the breast 64 with the x-ray system 10 in step 804. Subsequently, or alternatively, in step 808, the breast 64 is released, repositioned and recompressed to obtain an MLO image, e.g., a LE MLO image 811, of the breast 64. The compression of the breast 64 in either the LE CC image 806 or the LE MLO image 811 can be the first compression.

At any time after obtaining the LE CC image 806 and/or LE MLO image 811, in step 810 the LE CC image 806 (or LE MLO image 811) is analyzed by the user and/or CAD/controller 44 to determine the presence of any ROIs 812 and/or other triggering attributes, characteristics or findings in the breast 64 within the LE CC image 806 (or LE MLO image 811). If an ROI 812 and/or other triggering attribute(s), characteristic(s) or finding(s) in the breast 64 is found in the LE CC image 806, in step 820 the breast 64 is released, repositioned and recompressed to obtain an HE CC image 822 of the breast 64. In addition to, or as a substitute for the HE CC image 822, where the image analyzed for ROIs 812 is the LE MLO image 811, in step 820 the breast 64 is released, repositioned and recompressed in a second compression to obtain an HE MLO image 826 of the breast 64. As determined by the selection of the analyzed image, i.e., either the LE CC image 806 or the LE MLO image 811, the corresponding HE CC image 822 or HE MLO image 826 is determined to be obtained at the second compression.

After the acquisition of one or both of the HE CC image 822 and HE MLO image 826, in step 828 one of the LE CC image 806 and HE CC image 822 or the LE MLO image 811 and the HE MLO image 826 are registered to one another using a suitable registration algorithm to form a registered LE image 834. In step 836 the registered LE image 834 is recombined with the corresponding HE CC image 822 or HE MLO image 826 to form a recombined or reconstructed 2D or 3D image 838 clearly presenting the location and other characteristics of the ROI(s) 812 and/or other triggering attributes, characteristics or findings in the breast 64 for diagnostic review by the user and/or reviewing physician.

In the method 800 of FIG. 8, the initial LE CC image 806 and/or LE MLO image 811 can be obtained on one breast 64. While the analysis of the LE CC image 806 and/or LE MLO image 811 is performed in step 810, the x-ray system 10 can additionally be operated in a similar manner to obtain a LE CC image and a LE MLO image of the other breast (not shown) in order to enable the overlap or interleaving of the screening and diagnostic imaging procedures performed on each breast 64 using the method 800, or any other disclosed method. This significantly shortens the time required for the screening and potential diagnosis of each breast 64.

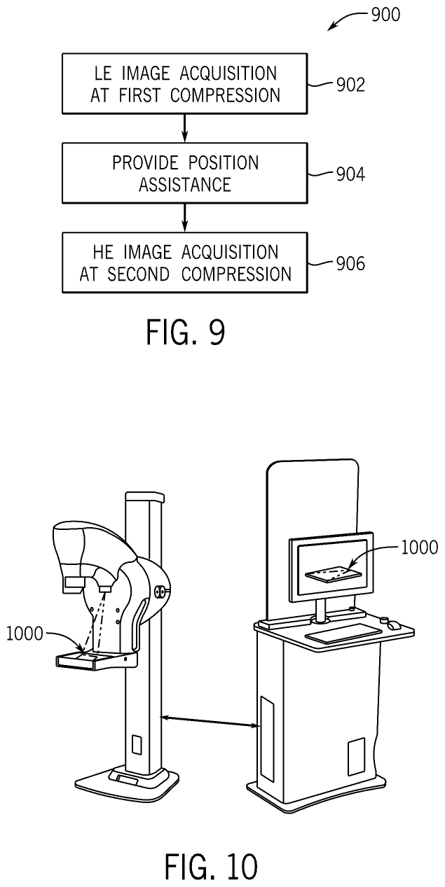

Referring now to FIGS. 9 and 10, in still a further exemplary embodiment of the invention, as an addition to any of the prior described methods 300-800, as illustrated in exemplary method 900 between the step 902 of acquiring the LE image at the first compression on the x-ray system 10 and the step 904 of obtaining the HE image at the second compression on the x-ray system 10, the methods 300-800 can include a position assistance step 906. In this step 906, which can be automatically employed by the x-ray system 10 or selectively employed by the user through the user interface 56 of the x-ray system 10, the x-ray system 10 provides an indication 1000 (FIG. 10) to the user regarding the compressed position of the breast 64 during the acquisition of the LE image for use a guide for the positioning of the breast 64 for the acquisition of the HE image. The indication 1000 can take any suitable form, such as representation of the breast 64 position from the LE image acquisition superimposed on a camera image of the breast 64 being positioned for the acquisition of the HE image present on the display 56, or a visible outline or similar shape of the breast 64 position from the LE image acquisition projected onto the detector 18 and the breast 64 being positioned for the acquisition of the HE image. The position of the indication 1000 relative to the actual location of the breast 64 being positioned for the acquisition of the HE image enables the user to closely approximate the positions of the breast 64 in each of the LE image and HE image acquisitions, thereby enhancing the quality of the registration and/or recombination of the LE and HE images to form the recombined or reconstructed 2D or 3D images for diagnostic purposes.

With regard to the recombined or reconstructed 2D or 3D image formed from the LE image(s) and the HE image(s) pursuant to any of the embodiments of the methods disclosed herein, the recombination can be performed by a suitable artificial intelligence (AI) or algorithm trained for this purpose. In one exemplary embodiment the recombination algorithm is a three-compartment breast (3CB) decomposition algorithm, such as disclosed in Laidevant A. D., Malkov S., Flowers C. I., Kerlikowske K., Shepherd J. A., “Compositional breast imaging using a dual-energy mammography protocol,” Med. Phys. 37(1), 164-174 (2010), which is incorporated herein by reference in its entirety for all purposes. The 3CB algorithm utilizes the LE and HE images obtained using the dual-energy mammogram method described herein with the breast thickness information to obtain three selective material images (i.e., water, lipid, and protein) Based on the material specific images, biologically relevant image analysis in the context of tumor malignancy can be conducted. Indeed, malignant tumors usually have different water/lipid/protein signature than benign ones. With this information on the types of tissue present in the ROI, the controller 44 can provide information to the user and/or reviewing physician regarding the ROI such as the determination the types and amounts of tissue present, such as to distinguish cysts from other lesion/tissue types within the breast 64, and/or a probability score that the ROI is a cyst and/or a score or value corresponding to the potential malignancy of the ROI based on the determined types and amounts of tissue types in the ROI, which can be presented on the display 50 in conjunction with the recombined or reconstructed 2D or 3D image.

In any of the aforementioned and described embodiments, the LE image utilized for the ROI analysis and or analysis for other triggering attributes, characteristics or findings in the breast 64 by the CAD system/controller 44 can be either one or more LE CC images, or one or more LE MLO images. Further, the one or more LE CC images or one or more LE MLO images being analyzed can be obtained by the x-ray system 10 in a 2D (FFDM) ( ) or 3D (DBT) screening imaging procedure being performed on a patient.

Finally, it is also to be understood that the system 10 may include the necessary electronics, software, memory, storage, databases, firmware, logic/state machines, microprocessors, communication links, displays or other visual or audio user interfaces, printing devices, and any other input/output interfaces to perform the functions described herein and/or to achieve the results described herein. For example, as previously mentioned, the system may include at least one processor and system memory/data storage structures, which may include random access memory (RAM) and read-only memory (ROM). The at least one processor of the system 10 may include one or more conventional microprocessors and one or more supplementary co-processors such as math co-processors or the like. The data storage structures discussed herein may include an appropriate combination of magnetic, optical and/or semiconductor memory, and may include, for example, RAM, ROM, flash drive, an optical disc such as a compact disc and/or a hard disk or drive.

Additionally, a software application that adapts the controller to perform the methods disclosed herein may be read into a main memory of the at least one processor from a computer-readable medium. The term “computer-readable medium”, as used herein, refers to any medium that provides or participates in providing instructions to the at least one processor of the system 10 (or any other processor of a device described herein) for execution. Such a medium may take many forms, including but not limited to, non-volatile media and volatile media. Non-volatile media include, for example, optical, magnetic, or opto-magnetic disks, such as memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, a RAM, a PROM, an EPROM or EEPROM (electronically erasable programmable read-only memory), a FLASH-EEPROM, any other memory chip or cartridge, or any other medium from which a computer can read.

While in embodiments, the execution of sequences of instructions in the software application causes at least one processor to perform the methods/processes described herein, hard-wired circuitry may be used in place of, or in combination with, software instructions for implementation of the methods/processes of the present invention. Therefore, embodiments of the present invention are not limited to any specific combination of hardware and/or software.

It is understood that the aforementioned compositions, apparatuses and methods of this disclosure are not limited to the particular embodiments and methodology, as these may vary. It is also understood that the terminology used herein is for the purpose of describing particular exemplary embodiments only, and is not intended to limit the scope of the present disclosure which will be limited only by the appended claims.

We claim:

1. A method for determining the need for generation of enhanced diagnostic images of a patient, the method comprising the steps of:
a. providing an imaging system comprising:
i. a radiation source operable at to emit radiation at multiple energy levels, a detector alignable with the radiation source, the detector having a surface on which an object to be imaged is adapted to be positioned; and
ii. a controller operably connected to the radiation source and the detector to control the operation of the radiation source and detector to generate image data in an imaging procedure performed by the imaging system, the controller including a central processing unit and interconnected database for processing the image data from the detector to create images, a display operably connected to the controller for presenting information to a user, and a user interface operably connected to the controller to enable user input to the controller;
b. positioning the object on the surface between the radiation source and the detector;
c. acquiring one or more low energy (LE) images of the object;
d. analyzing the one or more LE images to locate one or more triggering attributes, characteristics or findings within the object;
e. optionally acquiring one or more additional images of the object, the one or more additional images acquired with radiation having an energy level different than employed for acquiring the one or more LE images if one or more triggering attributes, characteristics or findings are located in the object; and
f. optionally processing the one or more LE images and the one or more additional images to form one or more enhanced images.

2. The method of claim 1, wherein the step of optionally acquiring the one or more additional images of the object comprises acquiring one or more high energy (HE) images of the object.

3. The method of claim 2, wherein the step of optionally processing the one or more LE images and the one or more additional images to form one or more enhanced images comprises processing the one or more LE images and the one or more HE images to form one or more enhanced 2D images, one or more enhanced 3D images, or combinations thereof.

4. The method of claim 3, wherein the imaging system is a mammography imaging system including a gantry supporting the radiation source and the detector and operably connected to the controller, a compression plate disposed on the gantry and movable with respect to the radiation source and the detector, wherein the object is a breast, wherein the one or more triggering attributes, characteristics or findings within the object are one or more regions of interest (ROIs) within the breast and wherein the step of positioning the object on the surface between the radiation source and the detector comprises the steps of:
a. placing the breast on the surface of the detector; and
b. moving the compression plate to compress the breast at a first compression between the compression plate and the surface.

5. The method of claim 4, further comprising the steps of:
a. moving the compression plate to decompress the breast from the first compression between the compression plate and the surface after acquiring the one or more LE images of the breast; and
b. moving the compression plate to recompress the breast at a second compression between the compression plate and the surface.

6. The method of claim 5, wherein the step of acquiring one or more LE images of the breast comprises acquiring one or more LE images of the breast in a cranial-caudal (CC) or mediolateral oblique (MLO) view at the first compression, and wherein the method further comprises the step of optionally acquiring one or more HE images of the breast in a CC or MLO view at the second compression after moving the compression plate to recompress the breast at a second compression between the compression plate and the surface.

7. The method of claim 6, wherein the one or more LE images at the first compression and the one or more HE images at the second compression are acquired in the same CC or MLO view, wherein the method further comprises the step of registering the one or more LE images to the one or more HE images to form one or more registered LE images prior to processing the one or more registered LE images and the one or more HE images to form one or more enhanced 2D images, one or more enhanced 3D images, or combinations thereof.

8. The method of claim 4, wherein the step of acquiring the one or more LE images comprises acquiring the one or more LE images in a 2D acquisition procedure.

9. The method of claim 8, wherein the step of acquiring the one or more HE images of the breast comprises:
a. acquiring the one or more HE images in a 2D acquisition procedure; or
b. acquiring the one or more HE images in a 3D acquisition procedure.

10. The method of claim 4, wherein the step of acquiring the one or more LE images comprises acquiring the one or more LE images in a 3D acquisition procedure.

11. The method of claim 10, wherein the step of acquiring the one or more HE images of the breast comprises:
a. acquiring the one or more HE images in a 2D acquisition procedure; or
b. acquiring the one or more HE images in a 3D acquisition procedure.

12. The method of claim 4, further comprising the step of presenting a positioning indicator illustrating a position of the breast in the first compression prior to moving the compression plate to recompress the breast at the second compression.

13. The method of claim 1, wherein the step of analyzing the one or more LE images is performed manually through the user interface.

14. The method of claim 1, wherein the imaging system includes a collimator mounted to the radiation source, and wherein the step of acquiring one or more additional images of the object comprises the steps of:
a. adjusting the collimator on the radiation source; and
b. acquiring the one or more additional images.

15. The method of claim 1, wherein the imaging system further comprises a computer aided detection (CAD) system operably connected to the controller and configured to analyze images created by the controller and locate the one or more triggering attributes, characteristics or findings within the object, and wherein the step of analyzing the one or more LE images is performed automatically by the CAD system.

16. A mammography system comprising:
a. a radiation source operable at to emit radiation at multiple energy levels, a detector alignable with the radiation source, the detector having a surface on which an object to be imaged is adapted to be positioned;
b. a controller operably connected to the radiation source and the detector to control the operation of the radiation source and detector to generate image data in an imaging procedure performed by the imaging system, the controller including a central processing unit and interconnected database for processing the image data from the detector to create images;
c. a display operably connected to the controller for presenting information to a user; and
d. a user interface operably connected to the controller to enable user input to the controller;
wherein the controller is configured to acquire one or more low energy (LE) images of the object with the radiation source and the detector, to optionally acquire one or more additional images of the object after a determination of one or more triggering attributes, characteristics or findings in the object within the one or more LE images, the one or more additional images acquired with radiation having an energy level different than employed for acquiring the one or more LE images if one or more triggering attributes, characteristics or findings are located in the object, and to optionally process the one or more LE images and the one or more additional images to form one or more enhanced images.

17. The mammography system of claim 16, wherein the controller is configured to acquire the one or more LE images and the one or more additional images in the same view of the object and to register the one or more LE images to the one or more additional images to form one or more registered LE images prior to processing the one or more registered LE images and the one or more additional images to form one or more enhanced 2D images, one or more enhanced 3D images, or combinations thereof.

18. The mammography system of claim 16, wherein the controller is configured to acquire the one or more LE images in one of a 2D acquisition procedure or a 3D acquisition procedure and to acquire the one or more additional images in one of a 2D or a 3D acquisition procedure.

19. The mammography system of claim 16, wherein the mammography system further comprises a computer aided detection (CAD) system operably connected to the controller and configured to perform the determination of one or more triggering attributes, characteristics or findings in the object within the one or more LE images.

20. The mammography system of claim 16, wherein the object is a breast, and wherein the one or more triggering attributes, characteristics or findings in the object comprise one or more ROIs in the breast.
